SEQUENCE LISTING (2 sequences)

SEQ ID NO: 1 - protein, 487 aa, Aranicola proteolyticus
  Zn binding site (192)..(192)
  Zn binding site (196)..(196)
  Zn binding site (323)..(323)
MQSTKKAIEITESSLAAASSAYNAVDDLLHYHERGNGIQVNGKDSFSTEQAGLFITRENQTWNGYKVFGQPVKLTFSFPDYKFSSTNVAGDTGLSKFSAEQQQQAKLSLQSWSDVANITFTEVGAGQKANITFGNYSQDRPGHYDYDTQAYAFLPNTIYQGQNLGGQTWYNVNQSNVKHPASEDYGRQTFTHEIGHALGLSHPGDYNAGEGNPTYRDASYAEDTREFSLMSYWSETNTGGDNGGHYAAAPLLDDISAIQHLYGANQTTRTGDTVYGFNSNTGRDFLSTTSNPQKVIFAAWDAGGNDTFDFSGYTANQRINLNEKSFSDVGGLKGNVSIAAGVTIENAIGGSGNDVIVGNAANNVLKGGAGNDVLFGGGGADELWGGAGKDTFVFSAVSDSAPGASDWIKDFQKGIDKIDLSFFNQGAQGGDQIHFVDHFSGAAGEALLSYNASNNVSDLALNIGGHQAPDILVKIVGQVDVATDFIV

SEQ ID NO: 2 - DNA, 2481 bp, Aranicola proteolyticus
  misc_feature (402)..(402): n is a, c, g, or t
cgagcgcgaaagacccggaaggcacgaggtgattagtcaaaaaaagaaaatgttattcctgcgggaactaaaaagtaccggcggctaataataaagagttattaatctataacgctttagccaaatttaacttttagccgtctaaatcccagcacgattcgcttggctctgcaggccgcatttttgttggagtttgttaccaactcatggcatttaagtttcattaatattgtaaataatgcaaaaaaccagcataaatccccttcgtaacgataataaatggctgattattttatgtgcagttttacaccgctgcctataattggaatcgattaccatttatggtggtaatcttatttgctgatatatatgcattaattctctctaacacactgccggtancggcgcataaactccttcccgtaagcgtgcggttcgttctccgtggcttcctggcaggttatgtctatctgtctgattgaaaccaatcagctaatgagtggaatcgaaccaatgcaatctactaaaaaggcaattgaaattactgaatccagccttgcggctgcgagctccgcttacaatgcagtagatgatttgctgcattatcatgagcgaggcaacgggattcaggttaatggcaaggactcattttctaccgaacaagccgggctgtttattacccgcgagaaccaaacctggaacggttataaagtttttggccaaccggttaaattaacgttctctttcccggattataaattctcttccaccaacgtcgccggcgataccggactgagcaaattcagcgcggaacagcagcagcaggctaagctgtcgctgcagtcctggtctgacgtggccaatatcacctttaccgaagttggtgccggccagaaggccaatatcaccttcggtaactacagccaggatcgtcccggccattatgactacgatacccaggcttacgccttcctgccgaacaccatttatcagggccaaaacctgggcgggcagacttggtacaacgtcaaccagtccaacgtgaaacatccggccagcgaagactacggccgccagacctttacccacgagattggccatgcgttgggcttgagccatccgggcgattacaacgccggcgaaggcaacccgacttacagagatgccagctacgccgaagatactcgtgagttcagcctgatgagttactggagcgaaaccaacaccggtggcgacaacggcgggcactacgctgcggcgccactgctggatgacatttccgctattcagcatctgtatggtgccaaccagaccacccgtaccggcgataccgtgtatggcttcaactcaaataccggacgtgacttcctcagtaccaccagcaatccgcaaaaagtgatctttgcggcctgggatgcgggtggtaatgacaccttcgatttctccggttacaccgctaaccagcgtattaatctgaacgagaaatctttctccgacgtgggtgggctgaaaggcaacgtgtccattgccgcaggtgtgaccatcgagaacgcgattggcggttcaggcaatgacgtgatcgtcggcaatgcggccaacaacgtgctgaaaggtggcgcgggcaacgacgtgctgttcggcggtggtggggctgatgagctgtggggcggtgcgggcaaagacacctttgtcttctctgcggtcagcgattctgcgccgggtgcctccgactggatcaaggatttccagaaaggcatcgataaaatcgacctgtcattcttcaatcagggcgcgcagggtggcgatcagatccacttcgtcgatcatttcagtggcgcagcgggcgaagccttgctgtcttacaatgcgtcgaataacgtcagcgatctggccctgaatatcggcggccatcaggccccggacatcctggtgaagatcgtcggccaggttgatgtcgccactgactttatcgtttaacagtgcaggtgctaacgcccggcgccggttggccgggcgttatacaggagacgatatgaagggcagcttagcgcacgccgccttagtggcaggcggcatgatggttacgggggcagttatggccagcagtttggttcttcccagcgcgcaatcattggcggggcaatggctggtcgccaatgccgaacaacaatgtcagattgagtttttggccggtgaacagagtgaaatcaacggctactcattggttgatcggcagcactgtttggaaaaggtgttaaccgccgaggtggtcggttggcgccctgcaccggacggcatcgctttgctgcgggcggatggcagtacgctggcgttcttctcgcgcgatggcgatatttaccgcaaccagcttggcgcggatgacggactgacgctgaaagcgctggtataacaacagcgggttcggcagtcgaacccgccctgagcagccttacagatacagcgaacgtacgatcaggaaat

(19) United States
(12) Patent Application Publication
(10) Pub. No.: US 2010/0278806 A1
(43) Pub. Date: Nov. 4, 2010

(54) Composition Containing Arazyme for the Prevention and Treatment of Arthritis

(75) Inventors: Ho-Yong Park, Daejeon (KR); Kwang-Hee Son, Daejeon (KR); Dong-Ha Shin, Daejeon (KR); Kyu-Shik Jeong, Daegu (KR)

Correspondence Address: GREENLEE SULLIVAN P.C., 4875 PEARL EAST CIRCLE, SUITE 200, BOULDER, CO, 80301, US

(73) Assignee: Insect Biotech Co., Ltd., Daejeon (KR)

(21) Appl. No.: 12/812,388

(22) PCT Filed: Jan. 9, 2009
(86) PCT No.: PCT/KR09/00147

§ 371 Date: Jul. 9, 2010

(30) Foreign Application Priority Data
Jan. 11, 2008 (KR) 10-2008-0003502

(51) Int. Cl. A61K 38/48; A61P 19/02

(52) U.S. Cl. 424/94.63

(57) ABSTRACT

Disclosed herein is a composition containing arazyme as an active ingredient for prevention and treatment of arthritis. More specifically, arazyme produced from Aranicola proteolyticus of the present invention may be useful as a composition for prevention and treatment of arthritis by inhibiting the expression of TNF-α which is an inflammation-inducing factor and preventing the loss of proteoglycan and collagen from joint cartilage to inhibit the progression of arthritis and protect joints.

### BACKGROUND OF THE INVENTION

1. Field of the Invention

The present disclosure relates to a composition containing arazyme as an active ingredient for prevention and treatment of arthritis.

2. Description of the Related Art

Arthritis refers to a disorder of the joint. Arthritis may be in various forms from minor to serious impairments and may not always worsen. Arthritis-related diseases are the most common degenerative intractable diseases, which 12% of total global population suffers from. About 2 million or more people in Korea are suffering from such diseases. Arthritis is a general term for indicating symptoms of all the musculoskeletal system caused by inflammatory changes in musculoskeletal and connective tissues. The disease is characterized by chronic inflammation causing permanent damages in tissues, deformity and degeneration, and having troubles in joint, bone, cartilage or the spinal cord (Hofbause, L. C., et. al., Arthritis and Rheumatism 44:253-259, 2001).

Arthritis is classified into degenerative arthritis (osteoarthritis), rheumatoid arthritis, ankylosing spondylitis, reactive arthritis, psoriatic arthritis, systemic lupus erythematosus, polymyositis, polymyalgia rhematica, etc.

Osteoarthritis or wear-and-tear arthritis, also known as degenerative arthritis, is the most common of all arthritis-related diseases, usually occurring in middle-aged and elder people. Osteoarthritis a type of arthritis that affects joints of the spine and lower limbs (hip joints, knees, and foot joints), and more than 60% of people in their 60's or 70's have the disease. Osteoarthritis is a common disease that affects people as they get older. Although the disease is common in people who are over 60 years of age, it occurs more often in men than in women less than 45 years of age and it is more common in women than in men more than 55 years of age. Osteoarthritis may largely be classified into primary and secondary depending on the cause. Osteoarthritis showing symptoms by worn-out joint cartilage without a clear cause, that is, showing degenerative changes are referred to as primary osteoarthritis which often occurs in women. The exact cause and etiology of primary osteoarthritis is unclear. The cause of secondary osteoarthritis may be largely classified into joint damage, abnormality of cartilage matrix, deformity of subchondral bone, etc.

Degenerative arthritis is a chronic arthritis with the frequency increasing with age, and knee osteoarthritis, in particular, is a disease with a high incidence rate. There is a report that for Koreans who usually lead a sedentary lifestyle, the knee is bent in an O shape which increases pressure abnormally on the inner portion of knee, and the possibility of having osteoarthritis is increased in middle age. When this inflammation continues, the atrophy of surrounding muscles may cause joints to be deformed, resulting in various social losses such as medical expenses, reduced social life of elderly people, etc.

Rheumatoid arthritis is the most common type of inflammatory arthritis, and 1% to 2% of the population is suffering from the disease. Rheumatoid arthritis is caused by inflammation of synovial joints and may occur to anyone who is 10 years or older. However, the disease is more common in women and often occurs at 30 to 50 years of age.

Ankylosing spondylitis is a disease that causes inflammation of the joints between the spine and pelvis. It sometimes occurs in the hip. The disease affects men more than women by a ratio about of 3:1, and most often appears at ages between 20 and 40. Ankylosing spondylitis is 20 times more common in individuals whose siblings or parents have the disease.

Systemic lupus erythematosus is one of several diseases known as Connective Tissue Diseases. Systemic lupus erythematosus is similar to rheumatoid arthritis in that it is an autoimmune disease. However, the disease occurs much less frequently and its symptoms are less severe than rheumatoid arthritis. 90% of people with this disease are young females in their 20's and 40's.

Pharmacologic therapy, exercise therapy, operation therapy, etc. are used for treatment of arthritis. Among them, drugs such as analgesics, adrenal cortex hormone preparations, non-steroid anti-inflammatory drugs, etc., which usually reduce inflammation and alleviate pain, are used as pharmacologic therapy, and no specific therapeutic drug is available. Although one ingredient constituting joint cartilage is sold as a health supplementary food to exhibit its efficacy, more researches are needed because the therapeutic effects are not sustained.

Although analgesics alleviate pain, there is no anti-inflammatory action in them. Analgesics except for Tylenol may induce drowsiness and constipation. Therefore, there is little effect for joint stiffness or edema.

Although non-steroid anti-inflammatory drugs out of anti-inflammatory drugs reduce inflammation like steroids, the drugs are quite different from steroids in terms of mode of pharmacological action or potential side effects. Non-steroid anti-inflammatory drugs are especially beneficial for removal of stiffness or edema caused by pain as well as inflammation. However, when non-steroid anti-inflammatory drugs are internally used in overdose or for a long time, side effects such as bleeding of stomach wall, gastric ulcer, maldigestion, etc. occur.

Thus, the present inventors have performed research on effects of arazyme derived from Aranicola proteolyticus strain, revealed that as a result of induction of osteoarthritis in an animal model of Newzealand White Rabbits, known to induce lesions similar to those of human degenerative joint disease, and treatment with arazyme, the expression of TNF-α is inhibited and the loss of proteoglycan and collagen from joint cartilage is prevented to inhibit the progression of arthritis and protect joints, and made the present invention.

### SUMMARY OF THE INVENTION

One object of the present invention is to provide a composition and a health functional food both containing arazyme produced by Aranicola proteolyticus as an active ingredient for prevention and treatment of arthritis.

In order to achieve the objects, the present invention provides a preventative and therapeutic agent for arthritis containing arazyme as an active ingredient.

The present invention also provides a health functional food containing arazyme as an active ingredient for prevention and improvement of arthritis.

The present invention also provides a method for treating arthritis, including administering to an individual suffering from arthritis a pharmaceutically effective amount of arazyme.

The present invention also provides a method for preventing arthritis, including administering to an individual a pharmaceutically effective amount of arazyme.

The present invention also provides a use of arazyme in the preparation of a preventative and therapeutic agent for arthritis.

Furthermore, the present invention provides a use of arazyme in the preparation of a health functional food for prevention and improvement of arthritis.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph illustrating the measurement result of an expression level of TNF-α in plasma;

FIG. 2 is a group of pictures illustrating changes visually observed in condyle of femur:

M: medial side; and

Red arrow: eroded part,

FIG. 3 is a group of pictures illustrating changes visually observed in condyle of tibia;

FIG. 4 is a group of graphs illustrating the evaluation result of changes visually observed in condyles of femur and tibia with grades;

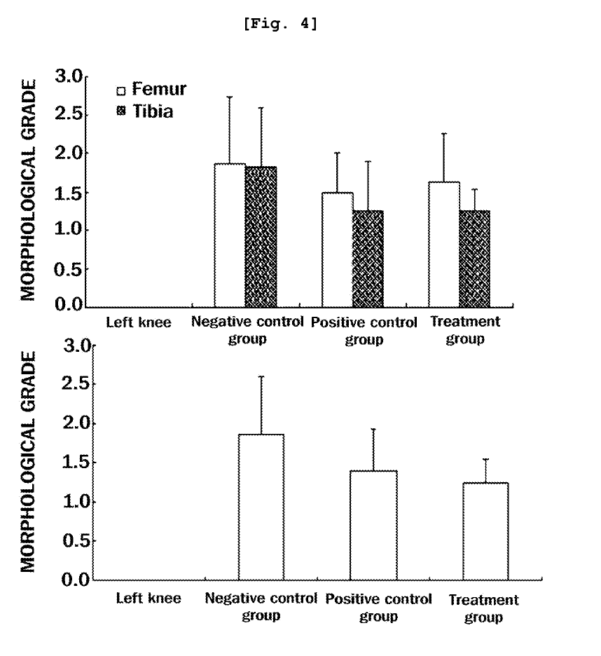

FIG. 5 is a group of pictures illustrating changes observed in tissue through staining of tissue samples with hematoxylin-eosin;

FIG. 6 is a group of pictures illustrating histopathological findings of effects of arazyme administration on osteoarthritis through staining with toluidine blue;

FIG. 7 is a group of pictures illustrating histopathological findings of effects of arazyme administration on osteoarthritis through staining with azan; and

FIG. 8 is a group of graphs illustrating scores of histopathological change of effects of arazyme administration on arthritis.

### DESCRIPTION OF THE PREFERRED EMBODIMENTS

Features and advantages of the present invention will be more clearly understood by the following detailed description of the present preferred embodiments by reference to the accompanying drawings. It is first noted that terms or words used herein should be construed as meanings or concepts corresponding with the technical spirit of the present invention, based on the principle that the inventor can appropriately define the concepts of the terms to best describe his own invention. Also, it should be understood that detailed descriptions of well-known functions and structures related to the present invention will be omitted so as not to unnecessarily obscure the important point of the present invention.

Hereinafter, terms used in the present invention will be defined.

As used herein, the term “arthritis” denotes all conditions under which various bacteria such as tubercle bacillus, etc. enter into the joints to cause inflammation on the joints.

As used herein, the term “prevention” denotes all behaviors that inhibit the formation of arthritis or delay the progression of arthritis by administration of a composition of the present invention.

As used herein, the terms “treatment” and “improvement” denote all the behaviors that improve or advantageously modify symptoms of arthritis by administration of a composition of the present invention.

As used herein, the term “administration” denotes the introduction of a predetermined composition of the present invention to an individual by any suitable method.

As used herein, the term “individual” denotes all animals, such as human, monkey, dog, goat, swine, rats, etc., which have an arthritis whose symptoms may be improved by administration of a composition of the present invention.

As used herein, the term “pharmaceutically effective amount” denotes an amount sufficient to treat a disease at a reasonable benefit or risky ratio applicable to a medical treatment, and this ratio may be determined depending on factors including the kind and severity of disease, drug activity, sensitivity, administration time, discharge ratio, administration duration, co-administered drugs, and other factors well known in the medical field.

Hereinafter, the present invention will be described in detail.

The present invention provides a preventive and therapeutic agent of arthritis containing arazyme as an active ingredient.

The arazyme of the present invention may be preferably prepared by a method including, but not limited thereto, the following steps:
1) culturing Aranicola proteolyticus strain to obtain a culture solution;
2) filtering the culture solution to obtain a supernatant; and
3) using resin to purify arazyme from the supernatant.

In the preparation method, Aranicola proteolyticus strain may be preferably used as an arazyme-producing microorganism, and Aranicola proteolyticus HY-3 deposited at KCTC (Korean Collection for Type Cultures) of KRIBB (Korea Research Institute of Bioscience and Biotechnology) on Jul. 29, 1996 (Accession No: KCTC 0268BP) may be more preferably used, but is not limited thereto.

Aranicola proteolyticus HY-3 is an aerobic Gram-negative bacterium that is separated from the intestine of Nephila clavata, 0.5 to 0.8 mm in size, in round shape, has mobility, and is positive to catalase but negative to oxidase (WO 01/57222).

Arazyme obtained by the above method may be preferably used, and arazyme obtained by the following method may be more preferably used. Basic materials for the culture of Aranicola proteolyticus may be preferably in medicine grade, which may more easily give a product of much higher purity than those when only purification is performed. After the culture, ammonium sulfate precipitation (or acetone precipitation) is performed, followed by centrifugation and filtration to recover arazyme. Most of the other proteins produced from the microorganism have different precipitation concentration from arazyme. After recovering arazyme, a first purification is performed by membrane filter to eliminate impurities and a final purification is performed using ultra filtration system to obtain pure arazyme. The obtained high concentrated arazyme solution is freeze-dried by a freeze dryer, resulting in arazyme powder which will be used.

Arazyme of the present invention can be produced by the following method, but is not limited thereto:
1) cloning DNA of a nucleotide sequence containing the coding region of arazyme into an expression vector;
2) introducing the expression vector cloned in step 1) into host cells;
3) selecting host cells transformed in step 2); and
4) obtaining arazyme expressed from the host cells selected in step 3).

In this method, the nucleotide sequence containing the coding region of arazyme of step 1) is preferably DNA represented by SEQ ID No: 2 or DNA hybridized with the DNA containing the nucleotide sequence represented by SEQ ID No: 2 under strict condition. The strict condition is determined during washing after hybridization. For example, the strict condition indicates washing at room temperature with 6×SSC and 0.5% SDS for 15 minutes, washing at 45° C. with 2×SSC and 0.5% SDS for 30 minutes and washing at 50° C. with 0.2×SSC and 0.5% SDS for 30 minutes and repeating the washing twice. More preferably, the strict condition indicates that washing is performed at higher temperature. Particularly, washing is performed by the same conditions as the above except that the last two washings are performed at 60° C. with 0.1×SSC and 0.1%. Such strict conditions may be determined or regulated by those skilled in the art. The expression vector herein is preferably the conventional Gram negative bacteria or Gram positive bacteria well known to those skilled in the art. A commercially available vector also may be used and it is more preferred for the vector to include a drug resistant gene for better screening. Any vector may be used as long as it does not affect arazyme gene.

In the method, the host cell of step 2) may be selected from the group consisting of bacteria such as E. coli and Bacillus subtilis and yeasts such as Saccharomyces cerevisiae, Candia and Phicia, but is not limited thereto.

In the method, the selection of the host cell transformed in step 3) is preferably performed by screening using the drug resistant gene introduced in the vector, or in combination with screening using Southern blotting or PCR, but is not limited thereto.

In the method, arazyme obtained in step 4) may include any one obtained by any protein purification method. For example, the protein purification may be preferably effected by column chromatography, filtration, ultra filtration, salting out, solvent precipitation, solvent extraction, distillation, immuno-precipitation, SDS-polyacryl amide gel electrophoresis, isoelectric point electrophoresis, dialysis or recrystallization, but is not limited thereto. Arazyme encoded by DNA may be obtained by the conventional protein expression systems well known to those skilled in the art. Arazyme may be also recovered and purified from cell culture which expresses arazyme.

Arazyme of the present invention is preferably one selected from the group consisting of the following proteins, but not limited thereto:

(a) a protein having the amino acid sequence represented by SEQ ID No: 1;

(b) a protein encoded by DNA containing the coding region of the nucleotide sequence represented by SEQ ID No: 2;

(c) a protein having an amino acid sequence mutated from the amino acid sequence represented by SEQ ID No: 1 with substitution, deletion, insertion and/or addition of one or more amino acids therein and functionally identical to the protein having the amino acid sequence represented by SEQ ID No: 1; and

(d) a protein encoded by DNA hybridized with the DNA containing the nucleotide sequence represented by SEQ ID No: 2 under the strict conditions and functionally equal to the protein having the amino acid sequence represented by SEQ. ID. NO: 1.

The hybridization under the strict conditions gives DNA having high homology in nucleotide sequence, suggesting that it is very much likely that the isolated protein has the protein functionally identical to arazyme. The nucleotide sequence having high homology indicates the nucleotide sequence having 70% or more homology with the nucleotide sequence represented by SEQ ID No: 2, preferably 80% or more, more preferably 90% or more and most preferably 95% or more homology with the nucleotide sequence represented by SEQ ID No: 2. In addition, the amino acid sequence having 70% or more homology with the amino acid sequence represented by SEQ ID No: 1, preferably 80% or more, more preferably 90% or more, and most preferably 95% or more homology with the amino acid sequence represented by SEQ ID No: 1 may be used. The rate of homology may be determined by the conventional algorithm selected by those skilled in the art. The hybridization may be performed by DNA-DNA hybridization under the strict conditions well known to those skilled in the art, and the strict condition in the hybridization, as mentioned hereinbefore, is determined during washing after hybridization (Hames and Higgins, Eds. Nucleic Acid Hybridization, IRL Press, U.K., 1985).

The present invention provides a preventive and therapeutic agent of arthritis containing arazyme as an active ingredient.

The arthritis includes degenerative arthritis, Rheumatoid arthritis, ankylosing spondylitis, reactive arthritis, psoriatic arthritis, Systemic lupus erythematosus, polymyositis, or polymyalgia rhematica.

In order to investigate effects of arazyme on osteoarthritis, osteoarthritis was induced through rupture of the cranial cruciate ligament of the right frontlimb using an animal model of Newzealand White Rabbits, known to induce lesions similar to those of human degenerative joint disease and then arazyme was administered.

In the present invention, blood was collected from the rabbit into which arazyme had been administered and serum was isolated from the blood to analyze TNF-α in the serum using ELISA (Quantikine®, R&D system). As a result, it was determined that a test group into which arazyme had been administered inhibited the expression of TNF-α which is involved in the initial inflammation process, compared to a control group. Therefore, it is determined that arazyme of the present invention has effects of inducing the onset of osteoarthritis by inhibiting the expression of TNF-α which is an inflammation-inducing factor.

In order to investigate the changes of joint cartilage of the rabbits into which arazyme had been administered, femur was separated from tibia, the surrounding muscles were removed, and then the surface of joint cartilage was observed. As a result, it was observed that a test group into which arazyme had been administered showed less cartilage damage than a control group. Therefore, it is determined that arazyme of the present invention exhibits efficacy against arthritis.

In order to observe the change of tissue and the degree of loss of collagen and proteoglycan, tissue sections of the separated femur and tibia were prepared, each stained with hematoxylin-eosin (H&E), azan, and toluidine blue, followed by observation with optical microscope. As a result, a test group into which arazyme had been administered all exhibited less damage of cartilage or less loss of proteoglycan and collagen than a control group. Therefore, because arazyme of the present invention exhibits damage of cartilage similar to that of celecoxib which is a non-steroid anti-inflammatory and weak loss of proteoglycan and collagen, it is determined that arazyme is useful for prevention and treatment of osteoarthritis.

It was determined through the results that the administration of arazyme of the present invention inhibited the progression of osteoarthritis in an animal model in which osteoarthritis had been induced and alleviated symptoms of the disease. It was confirmed that the intake of arazyme inhibited the expression of TNF-α which is an inflammation-inducing factor and prevented the loss of proteoglycan and collagen from joint cartilage. Therefore, arazyme of the present invention may be used for prevention and treatment of arthritis, resulting in inhibiting the progression of osteoarthritis and protecting bone joints.

The preventive and therapeutic agent of arthritis of the present invention may contain, in addition to arazyme, one or more active ingredients having the same or similar function to arazyme. For administration, pharmaceutical preparations may be prepared by including one or more pharmaceutically acceptable carriers in addition to an active ingredient. The pharmaceutically acceptable carrier may be used by including saline solution, sterile water, Ringer's solution, buffered saline solution, dextrose solution, maltodextrin solution, glycerol, ethanol, and a mixture of one or more thereof, and other conventional additives such as antioxidants, buffers, bacteriostatic agents, etc. may be added if necessary. Preparation for injection, such as solutions, suspensions, emulsions, etc., powders, tablets, capsules, pills, granules, or liquids for injection may be also prepared by additionally adding diluents, dispersants, surfactants, binders, and lubricants. Furthermore, the composition may be preferably prepared in suitable forms for each disease or according to ingredients by suitable methods in the art and methods described in Remington's Pharmaceutical Science (Mack Publishing Company, Easton Pa., 18^{th}, 1990).

The composition for prevention and treatment of arthritis of the present invention can be administered orally or parenterally (for example, intravenous, subcutaneous, intraperitoneal or local injection) according to the desired method. The effective dosage of the composition may be determined according to weight, age, gender, health condition, diet, administration time, administration method, excretion rate and severity of a disease. The dosage of arazyme is 0.01 to 5000 mg/kg per day and preferably 0.01 to 10 mg/kg per day, and administration is performed once a day or preferably a few times a day.

The present invention also provides a method for treating arthritis, including administering a pharmaceutically effective amount of arazyme to an individual suffering from arthritis.

The present invention further provides a method for preventing arthritis, including administering a pharmaceutically effective amount of arazyme to an individual.

It is determined that because arazyme of the present invention inhibits the expression of TNF-α which is an inflammation-inducing factor and prevents the loss of proteoglycan and collagen from joint cartilage to inhibit the progression of arthritis and protect joints, it may be useful for prevention and treatment of arthritis by administering a composition containing the same to an individual.

The administration may include, in addition to arazyme of the present invention, one or more active ingredients having the same or similar function to arazyme.

The administration may be orally or parenterally effected, and may be used in the form of a general medicinal preparation.

The unit of administration may contain 1, 2, 3, or 4 times of the individual dosage, or ½, ⅓, or ¼ times. The individual dosage preferably includes an amount of an effective drug which is administered once, corresponding to 1, ½, ⅓, or ¼ times of a daily dose conventionally administered. The effective dose of the composition of the present invention is 0.01 to 5000 mg/kg and preferably 0.01 to 10 mg/kg, and administration may be performed once to 6 times a day.

The present invention provides a use of arazyme in the preparation of a preventive and therapeutic agent of arthritis.

It is determined that because arazyme of the present invention inhibits the expression of TNF-α which is an inflammation-inducing factor and prevents the loss of proteoglycan and collagen from joint cartilage to inhibit the progression of arthritis and protect joints, it may be useful as an active ingredient of a preventive and therapeutic agent of arthritis.

The present invention also provides a health functional food for prevention and improvement of arthritis containing arazyme as an active ingredient.

Furthermore, the present invention provides a use of arazyme in the preparation of a health functional food for prevention and improvement of arthritis.

When the Aranicola proteolyticus culture solution of the present invention or arazyme isolated from the same may be used as food additive, the Aranicola proteolyticus culture solution or arazyme isolated from the same may be added as it is or as mixed with other food components according to the conventional method. Arazyme is obtained and used by the same manner as described above. The blending amount of an active ingredient may be appropriately determined according to the purpose of use (prevention, health or treatment). In general, when food or beverage is manufactured, the Aranicola proteolyticus culture solution or arazyme isolated from the same may be preferably added in an amount of 0.01 to 10 parts by weight and more preferably 0.05 to 1 part by weight. However, for health and hygiene or long-term intake for health control, the amount may be equal to less than the range, but the active ingredient may be used in an amount equal to or more than the range because there is no problem in terms of safety issue.

There is no specific limitation in kind of the food. Examples of foods in which the material may be added include meats, sausages, breads, breads, chocolates, candies, snacks, confectionaries, pizzas, ramen, flour products, gums, dairy products including ice cream, soups, beverages, tea, drinks, alcohol drinks and vitamin complexes, etc., and all kinds of health food in general meaning are included.

The composition for health beverages of the present invention may additionally include various flavors or natural carbohydrates, etc., like other conventional beverages. The natural carbohydrates above described may be monosaccharides such as glucose and fructose, disaccharides such as maltose and sucrose, polysaccharides such as dextrin and cyclodextrin, and sugar alcohols such as xylitol, sorbitol, and erythritol. As a sweetening agent, natural sweetening agents such as thaumatin and stevia extract, and synthetic sweetening agents such as saccharin and aspartame may be used. In general, the content of the natural carbohydrate is generally about 0.01 to 0.04 g and preferably 0.02-0.03 g in 100 ml of the beverage composition of the present invention.

In addition to the ingredients mentioned above, the Aranicola proteolyticus culture solution of the present invention or arazyme separated therefrom may be added to in variety of nutrients, vitamins, minerals, flavors, coloring agents, pectic acid and its salts, alginic acid and its salts, organic acid, protective colloidal viscosifiers, pH regulators, stabilizers, antiseptics, glycerin, alcohols, carbonators which used to be added to soda, etc. The Aranicola proteolyticus culture solution of the present invention or arazyme separated therefrom may be also added to natural fruit juice, fruit beverages, and fruit flesh addable to vegetable beverages. These ingredients can be used alone or in combination. The mixing ratio of these ingredients does not matter in fact, but may be generally selected within the range of 0.01 to 0.1 part by weight based on 100 parts by weight of the Aranicola proteolyticus culture solution of the present invention or arazyme separated therefrom.

Hereinafter, the present invention will be described in more detail with reference to the following examples, experimental examples, and preparation examples.

However, the following examples, experimental examples, and preparation examples are provided for illustrative purposes only, and the scope of the present invention should not be limited thereto in any manner.

### Example 1

### Preparation of Arazyme

In order to prepare arazyme, an active ingredient of the present invention, Aranicola proteolyticus HY-3 (KCTC 0268BP) was cultured in a culture medium (bacto-trypton 0.5%, yeast extract 0.5%, sodium chloride 0.1%, potassium chloride 0.05%, calcium chloride 0.02%, magnesium sulfate 0.02%) at 22° C. for 18 hours. The culture solution was filtered by membrane filtration (2 μm filter, Satorius, USA) to separate supernatant from the cells. The supernatant was concentrated by membrane filtration (10 kDa Membrane filter, Pall sept, PALL Corporation, USA). Because arazyme of the present invention basically has the characteristics of anion, the concentrated solution was purified by ion exchange resin (Sigma USA) using DEAE-cellulose (Sigma, USA) pre-treated with 50 mM tris-HCl buffer (pH 7.6) and gel filtration exchange resin using Sephadex G-75 (Sigma USA) pre-treated with 20 mM tris-HCl buffer (pH 7.6). The purified enzyme solution was electrophoresed on 10% SDS-PAGE (Sodium dodecyl sulfate-polyacrylamide gel), and band pattern was confirmed. As a result, arazyme of the present invention was identified as a monomer not having a subunit and showed the band of approximately 51.5 kDa. However, the Aranicola proteolyticus HY-3 strain (KCTC 0268BP) may be cultured in various commercial media to produce arazyme and the culture solution thereof may be separated and purified by various methods. Arazyme of the present invention has the amino acid sequence represented by SEQ ID No: 1 and the nucleotide sequence represented by SEQ ID No: 2 encoding the same.

### Example 2

### Arrangement of Experimental Animals

<2-1> Breeding of Experimental Animals

For the experiment, the present inventors used NewZealand White Rabbits (Orient Co., Ltd., Seoul, Korea). When purchased, the experimental animals were 9 week old, and their weights were in the range of 1.2 to 1.5 kg. The animals were 15 weeks old when the administration for experiment began, and their weights were in the range of 2.1 to 2.7 kg at that time. After purchased, the animals were visually inspected and adapted for 7 days in an animal laboratory. During the adaptation, general symptoms were observed and only healthy animals were selected for the experiment.

The animals were adapted and raised in an animal laboratory of Department of Pathology, College of Veterinary Medicine, Kyungpook National University, equipped with an automatic temperature/humidity regulator by which temperature was set at 22±2° C. and the relative humidity was regulated to be 55±10% and light interval was set at 12 hours (light on at 09:00 and light off at 21:00). Any breeding environmental changes that might affect the experiment were not detected. During the whole experimental period, the temperature/humidity of the animal laboratory was regulated by the automatic temperature/humidity regulator and the environmental conditions were checked regularly (once every three months). From environmental checkup, any changes that might affect the experiment were not detected. During the whole experimental period, an animal was accommodated in each breeding box with a steel mesh net on a floor installed with lustrous aluminum alloy and a removable fecal tray (width: 480, length: 610, height: 450 mm). For individual identification, both skin mark using an oil marker and group distinction cards at each breeding box were used. Solid fodder for rabbits (Agribrands Purina Korea, Inc.) was freely fed, and drinking water mixed with tap water and test material was freely given by a bottle.

<2-2> Induction of Arthritis in Experimental Animals

It is known that not only repetitive joint damage but also a lesion similar to that of human degenerative osteoarthritis is induced in a rabbit model with knee meniscus removed. A method for tearing the cranial cruciate ligament is used as a method for inducing osteoarthritis in rabbits (Hulth A. et al., Acta Orthp Scand. 41:522-530, 1970). In the present experiment, the cranial cruciate ligament of a rabbit was torn to induce the instability of joints and simultaneously medial meniscus was removed to induce osteoarthritis.

The cranial cruciate ligaments of right knees of all the experimental animals were torn to induce the instability of joints and medial meniscuses were simultaneously removed to induce osteoarthritis. Sharm-operations were performed on the left knees to be used as a control against administration of a test material. Tear of cranial cruciate ligament and medial meniscus removal were performed as follows. The animals were fasted for 6 to 12 hours prior to the operation and only drinking water was supplied. Subsequently, an anesthetic of Xylazine (Rompun, Bayer Korea Ltd.) at 1.25 ml/6 mg/kg and Zoletil50 (Virbac) at 0.2 ml/10 mg/kg was intramuscularly injected to anesthetize the animals. After the rabbits were anesthetized, skin and muscle fascia were torn apart from the inner part of knees of the rabbits, the joint capsule exposed due to rupture of the skin and knee was torn, and some portions of femur and tibia were exposed. After the exposure, the cranial cruciate ligament between femoral joint surface and tibial joint surface was cut and the medial meniscus was removed. After the surgery, the torn muscle fascia and skin were sutured. After the surgery, gentamicin (SamU Median Co., Ltd.), an antibiotic, was injected at 0.1 ml/10 mg/kg for 3 days to prevent the onset of infection in the surrounding tissues due to the surgery. In order to induce osteoarthritis effectively, all the animals in the test group were subjected to a forced exercise task using a treadmill for 5 min every day.

<2-3> Organization of Experimental Animals

4 to 5 experimental animals with the weight of 2.1 to 2.7 kg were used in each group and the experimental group was divided into 3 groups. Celecoxib (Pfizer Pharmaceuticals Korea Ltd.) was daily supplied to a positive control group at a concentration of 10 mg/kg/day in 250 to 300 ml of tap water, and a composition of the present invention was diluted at 250 mg/kg in 250 to 300 ml of tap water and daily fed to a test group. Celecoxib (Pfizer) used to the positive control group is a non-steroid anti-inflammatory drug usually prescribed to patients with osteoarthritis. Although it exhibits better therapeutic effects in patients with osteoarthritis than traditional non-steroid anti-inflammatory drugs, celecoxib is used for treatment of osteoarthritis due to less induction of peptic ulcer (Loewen P S. focus on clinical aspects. 4:268-75, 2002). 250 to 300 ml of tap water was daily supplied to all the animals in a negative control group at an equal volume. A test material and 250 to 300 ml of tap water were daily fed to all the animals at an equal volume with the same administration route and administration method.

TABLE 1
Organization of Test groups

| | | Arthritis | Drug |
|---|---|---|---|
| Group | No. of animals | surgery | administration |
| Negative control group | 4 | Right knee | Tap water |
| Positive control group | 5 | Right knee | Celecoxib |
| Treatment group | 5 | Right knee | Arazyme |

In order to induce osteoarthritis effectively, all the animals in the experimental group were subjected to a forced exercise task using a treadmill for 5 min every day from the day 4 after the induction surgery of osteoarthritis, and arthrography was performed on both knee joints at 4 weeks, 8 weeks, and 12 weeks after administration of the test material to measure the changes of joint surface. An autopsy was performed at 12 weeks after administration of the test material to conduct serologic tests of blood samples through collection of blood and control effects of the test material on osteoarthritis were assessed through a histopathological test.

### Experimental Example 1

### Measurement of Effects of the Inhibition of Expression of TNF-α

The experimental animal was injected and anesthetized with xylazine and zoletil and subjected to laparotomy to collect blood samples from the saphenous vein, followed by centrifugation at 3000 rpm for 15 min to isolate the serum. TNF-α in the serum was analyzed by ELISA (Quantikine®, R&D system).

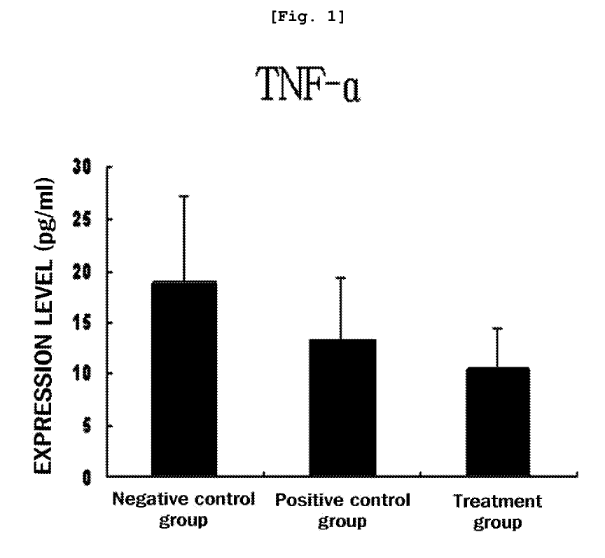

As a result of the measurement, a statistical significance was not shown based on the negative control group. However, the expression of TNF-α which is involved in the initial inflammation process was observed at a lower level than those of the positive control group and the test material group. The test material group exhibited the expression of TNF-α at a lower level than the negative control group. Therefore, it was determined that arazyme of the present invention inhibits the expression of TNF-α which is an inflammation-inducing factor to inhibit the onset of osteoarthritis (See FIG. 1).

### Experimental Example 2

### Visual Observation of the Changes of Joint Cartilage

The present inventors visually observed whether all the internal organs had abnormalities when the autopsy was performed, and then visually observed the changes of the surface of joint cartilage by separating both femurs from tibias and removing the surrounding muscles. During the visual observation of changes of joint cartilage, judgment standards on the lesion are listed in the following Table 2 (Shimizu C. et al., Long-term effects of hyaluronan on experimental osteoarthritis in the rabbit knee. 6:1-9, 1998).

TABLE 2
Evaluation of Grades by Visual Observation
of Arthritis-Induced Cartilage

| Grade | Related Standard |
|---|---|
| G0 | Normal joint surface |
| G1 | Weak fibrillation or weak discoloration of joint |
| | surface |
| G2 | Weak erosion of medial condyle and fibrillation of |
| | lateral condyle |
| G3 | Moderate necrosis and fibrillation of femoral condyle |
| G4 | Excessive erosion, apparent fibrillation, and |
| | formation of osteophyte |

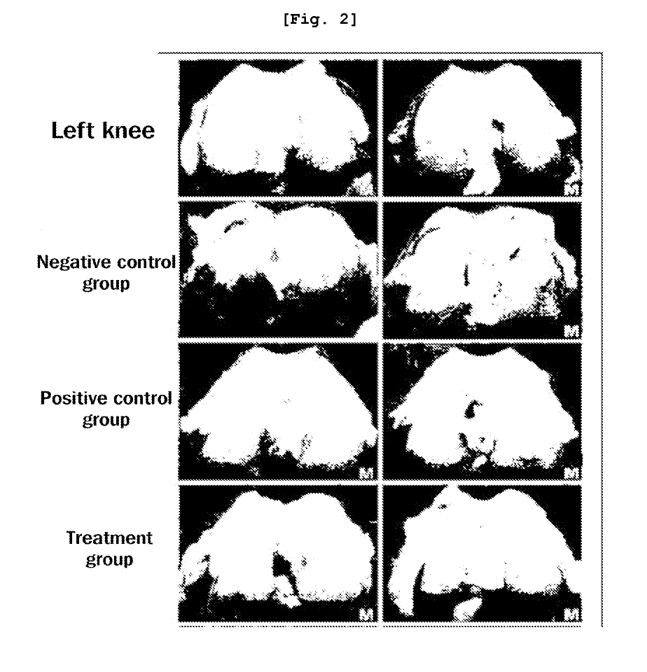

Femur and tibia were separated during the autopsy and the surrounding tissues were removed to perform a primary visual inspection of the changes of joint surface. As a result of observation, normal left knee joint surface had a smooth joint surface and was easily removed from the surrounding tissues. Conversely, it was observed that the osteoarthritis-induced right knee had a joint surface disrupted and thinly peeled off, and recessed marks were sometimes found. When femoral cartilage in the negative control group was visually observed, the severest lesion was able to be observed and cartilage was splited to produce a deep trough, or a moderate fibrillation and partial erosion of cartilage were observed. Although there was no clear difference between the groups into which a control drug and a test material had been administered from the visual observation, less cartilage damage was generally shown than in the negative control group. While the left tibia which had not been subjected to surgery had a normal smooth joint surface in case of the tibia joint surface, the joint surface of the osteoarthritis-induced right tibia was shown to be roughly disrupted, and its separation from the surrounding tissues was not easy, either (See FIG. 2).

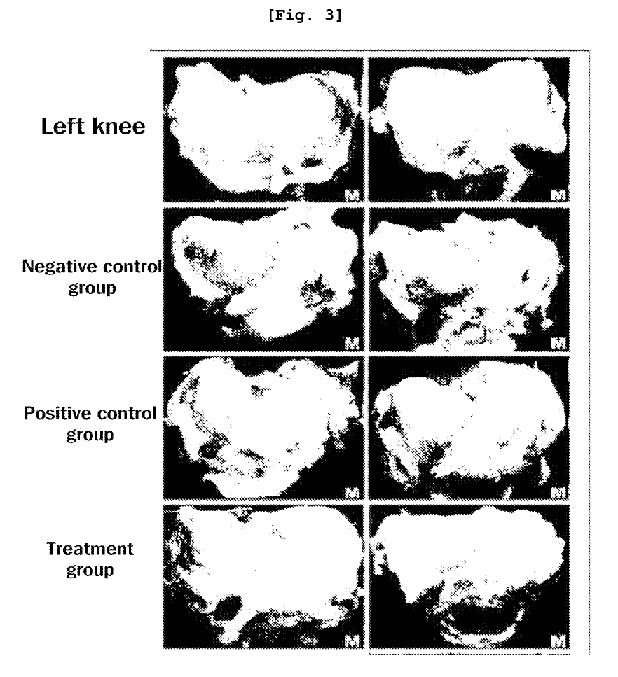

Although a moderate fibrillation and partial erosion of cartilage were observed in the negative control group during observation of joint surface of tibia, it was difficult to identify a difference between the positive control group and the test material group because generally similar lesions were observed in the groups. However, when the degrees of damage of joint surfaces of femur and tibia were differentiated, it was difficult to identify a difference between the groups except for the negative control group, but similar damage levels were observed from the positive control group and the treatment group (See FIG. 3).

### Experimental Example 3

### Histopathological Observation of Joint Cartilage

Each of the separated femur and tibia was fixed in 10% neutral buffered formalin. In order to cut tissues by a general sample preparation method, the separated bones were immersed in a decalcification solution containing EDTA for 3 months for decalcification, subjected to a general tissue treatment process, embedded in paraffin, followed by manufacture of a tissue sample of about 4 μm. The tissue sample was stained with hematoxylin-eosin (H&E) to observe the changes of tissues, stained with azan to observe the degree of loss of collagen fibers, and the degree of loss of proteoglycan was observed with optical microscope through staining with toluidine blue (Prizker K P, et al., Osteoarthritis Cartilage, 14:13-29, 2006).

When histopathological changes of joint cartilage are observed, judgment standards on the lesion are as follows. A Grade according to degree of damage of joint cartilage (See Table 3) and a step according to range of damage (See Table 4) were obtained and multiplied by each other to obtain a score (See Table 5) for scoring of the degree of damage of cartilage.

All the data in the test were expressed as average and standard error of each group. A statistical analysis was performed using a student t-test. When the p-value was less than 0.05, it was assessed to be significant.

TABLE 3
Evaluation of Histopathological Grades
on Osteoarthritis-Induced Cartilage

| Grade | Secondary Grade | Related Standard |
|---|---|---|
| Grade 0: | None | Normal Cartilage |
| Normal | | |
| Cartilage | | |
| Grade 1: | 1.0 Cartilage Cell | Matrix: Although the |
| Cartilage | Normal 1.5 Cell | surface layer is normal, |
| Surface | Death | there is edema or |
| Normal | | fibrillation |
| | | Observed Cell: |
| | | Proliferation and |
| | | hypertrophy of cartilage |
| | | cells |
| Grade 2: | 2.0 Fibrillation | Loss of proteoglycan from |
| Discontinuity | in the surface | the top ⅓ layer of the |
| of Cartilage | layer 2.5 Matrix | surface layer of |
| Surface | loss and peeling | discontinuous joint |
| | off in the surface | cartilage (Arrangement of |
| | layer | chondron disoriented) |
| Grade 3: | 3.0 Simple | Loss of proteoglycan from |
| Vertical | Vertical Fissure | the top ⅓ layer of joint |
| Fissure | 3.5 Complex | cartilage (Formation of a |
| | Vertical Fissure | new collagen) |
| Grade 4: | 4.0 Formation of a | Formation of cyst in the |
| Erosion | Separation | lost cartilage matrix of |
| | Formation Layer in | cartilage matrix |
| | the Surface Layer | |
| | 4.5 Formation of a | |
| | Concave in the | |
| | Transitional Layer | |
| Grade 5: | 5.0 Bone Surface | Formation of a hardened |
| Cartilage | Normal 5.5 Formation | tissue on the surface or |
| peeled off | of a Recovery Tissue | formation of a recovery |
| | on the Surface | tissue including fiber |
| | | cartilage |
| Grade 6: | 6.0 Formation of | The abnormally formed |
| Deformity | an osteophyte in | microfracture and repair of |
| | the joint boundary | bone remodeled joint |
| | 6.5 Formation of | surface |
| | an osteophyte in | |
| | the joint boundary | |
| | and the center | |

Grade: Depth of damage of joint cartilage

TABLE 4
Evaluation of histopathological stage
on osteoarthritis-induced cartilage

| Stage | % involvement (Surface, Site, Volume) |
|---|---|
| Stage 0 | State at which no damage is observed |
| Stage 1 | <10% |
| Stage 2 | 10-25% |
| Stage 3 | 25-50% |
| Stage 4 | >50% |

Stage = Range of damage of joint cartilage

TABLE 5
Evaluation of Pathological Scores on Arthritis-Induced Cartilage

Stage

| Grade | S1 | S2 | S3 | S4 |
|---|---|---|---|---|
| G1 | 1 | 2 | 3 | 4 |
| G2 | 2 | 4 | 6 | 8 |
| G3 | 3 | 6 | 9 | 12 |
| G4 | 4 | 8 | 12 | 16 |
| G5 | 5 | 10 | 15 | 20 |
| G6 | 6 | 12 | 18 | 24 |

Score = Grade × Stage (Calculated by multiplying the grade with the stage score)

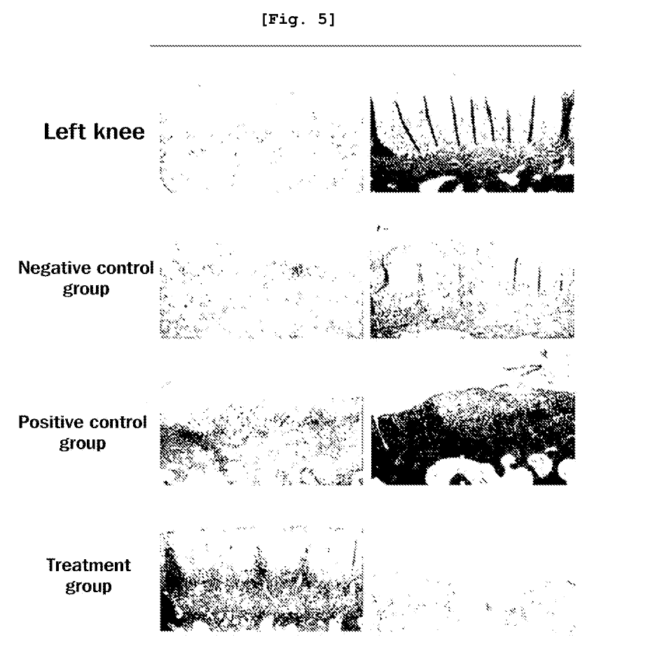

In the case of a normal left knee joint, the joint surface is smooth and the arrangement of chondrocyte and matrix is balanced. An observation revealed that three layers of joint cartilage (superficial zone, mid zone, and deep zone) were well differentiated and arranged, and the hypertrophy of chondron (cartilage cell including pericellular matrix) and proliferative change of cartilage cells were not observed. Except for chondrocyte, most of the joint cartilage (98˜99%) was composed of cartilage matrix and the main components of them were Type II collagen, proteoglycan, water, other proteins, glycoprotein, etc. In the case of a negative control group, it was observed that cartilage was splited into the mid zone and sometimes into the deep zone to form a vertical fissure or the cartilage was peeled off to expose a bone under the cartilage. The hypertrophy of chondron and excessive proliferation of chondrocyte were also observed around the cartilage which was damaged, splited, or peeled off, resulting in a wrinkled joint surface along with a general fibrillation on the surface of joints. In general, similar degrees of damage were shown in the positive control group and the treatment group. Although the formation of vertical fissure was observed into some deep zones, most of the vertical fissures were formed into sites corresponding to the mid zones from the surface, and the hypertrophy of chondron and the proliferation of chondrocyte were able to be observed along with the discontinuity of the surface of cartilage (See FIG. 5).

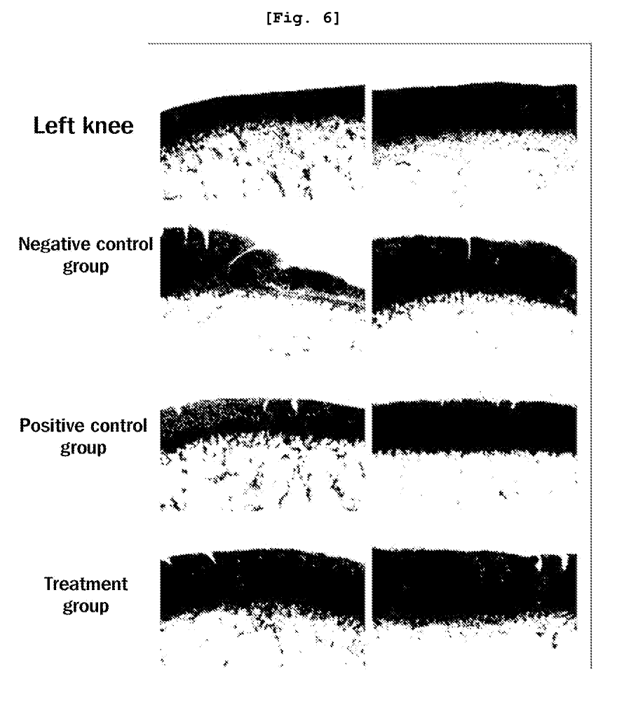

As in the negative control group in FIG. 6, sulfated glycosaminoglycan also known as proteoglycan is composed of a polymeric material which is a protein complex, and expressed as a blue color when stained with toluidine blue. Therefore, from a left normal knee joint stained with toluidine blue, a rich proteoglycan stained as dark blue was able to be observed. It was observed from the negative control group that the stainability with toluidine blue was reduced and proteoglycan was sometimes lost. Similar aspects were shown in the positive control group and the treatment group. Even in staining with toluidine blue, the loss of proteoglycan was apparently shown in the superficial zone, and it was observed that proteoglycan was lost into the mid zone (See FIG. 6).

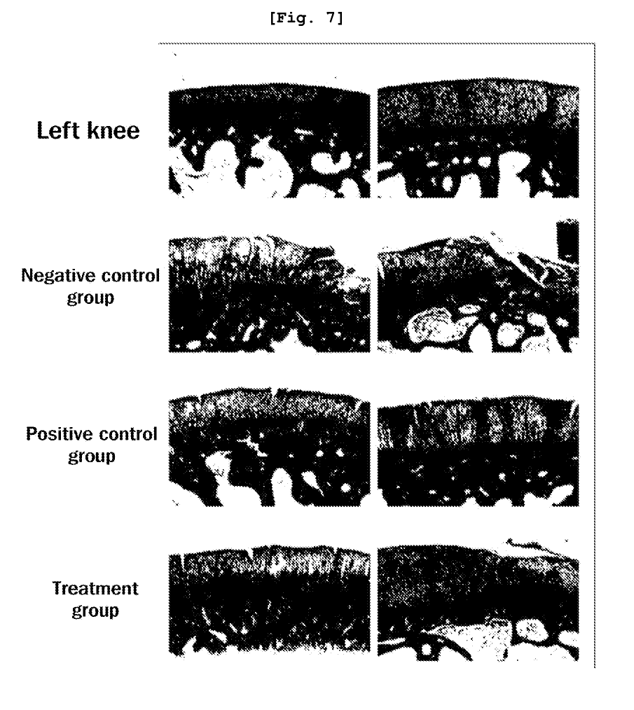

As illustrated in FIG. 7, collagen fibers sufficiently contained in the matrix of joint cartilage were stained with azan as blue color, and it was identified through this finding that a sufficient amount of collagen was normally present in the left knee joint. It was observed from the negative control group that the content of collagen in the matrix had been reduced compared to a normal state. A weak loss of collagen was generally observed through staining with azan in the positive control group and the treatment group (See FIG. 7).

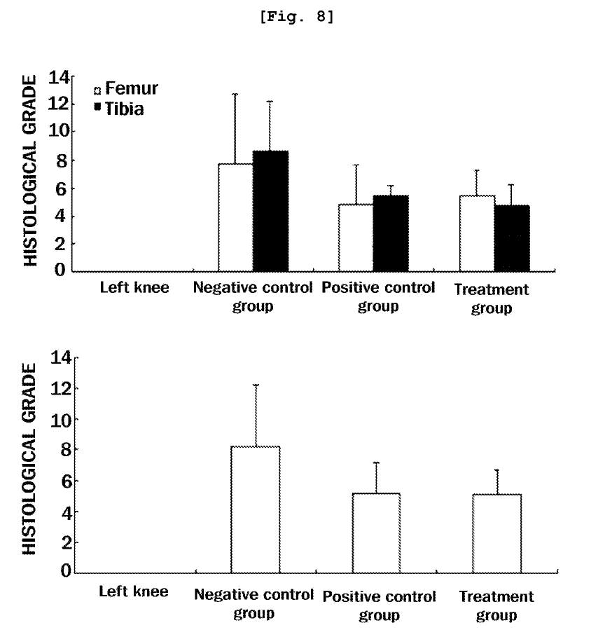

As in FIG. 8, as a result of grades on a histological observation based on this, similar grades were given to the positive control group and the treatment group. In verification of significance based on the negative control group, the p-value was not within the range of 0.05 or less. However, the p-values of the positive control group and the treatment group were 0.052 and 0.064, respectively in the positive control group (See FIG. 8).

### Experimental Example 4

### Acute Toxicity Experiment of Arazyme

In order to understand the acute toxicity of arazyme of the present invention, the following experiment was performed.

4 groups, each consisting of 4 Wistar-derived specific pathogen free (SPF) female rats (Orient Co., Ltd., Seoul, Korea), were bred in an animal laboratory under conditions of temperature 22° C.±3° C., humidity 55±10%, and illumination 12 L/12 D. After purchase, the rats were quarantined and adapted in the animal laboratory for 1 week. During the whole test period, 5 or less rats were accommodated in each polycarbonate cage (240 W×390 L×175 H mm). For individual identification, both skin mark using an oil marker and group distinction cards at each breeding box were used. Solid fodder for experimental animals (PMI Nutrition International, USA) was sterilized, and tap water was freely given by a water bottle.

Arazyme in the Examples was dissolved in sterilized water for injection at 0, 1250, 2500, and 5000 mg/kg, prepared at a dose of 10 ml/kg, and orally administered to the rats using zonde one. After 1 week adaptation, the rats were divided into 4 groups and arazyme was administered to 10 week-old rats at each concentration to observe general symptoms. Subsequently, an autopsy was performed after 24 hours in order to evaluate the toxicity of arazyme on actual organs.

After oral administration of arazyme into female rats, the behaviors, appearances, and functions of the animals were investigated in order to observe general symptoms exactly according to the administration of a test material and evaluate the toxicity.

As a result of the experiment, no abnormal symptoms in activity, walking, temperament and convulsion were observed in those animals after the administration of arazyme. No abnormal changes were observed in outward appearance including skin and hair, eyeball area, ears, reproductive organ, limbs, tail and in such functions as respiration, salivation, feces, vomiting, etc. From an autopsy performed 24 hours after the administration of arazyme, it was also confirmed that no abnormal symptoms were caused by arazyme in any organ. It is determined that estimated LD₅₀ of arazyme was at least 5000 mg/kg, which is a high concentration. No pathological symptoms were observed in actual organs such as liver, heart, lung and pancreas under microscope.

Hereinafter, preparation examples of the composition of the present invention will be exemplified.

### Preparation Example 1

### Preparation of Pharmaceutical Preparations

<1-1> Preparation of Powders

| | |
|---|---|
| Arazyme | 2 g |
| Lactose | 1 g |

The ingredients were mixed and filled into sealed packaging to provide powders.

<1-2> Preparation of a Tablet

| | |
|---|---|
| Arazyme | 100 mg |
| Corn starch | 100 mg |
| Lactose | 100 mg |
| Magnesium stearate | 2 mg |

The ingredients were mixed and tabletted according to a conventional tablet preparation method to provide a tablet.

<1-3> Preparation of a Capsule

| | |
|---|---|
| Arazyme | 100 mg |
| Corn starch | 100 mg |
| Lactose | 100 mg |
| Magnesium stearate | 2 mg |

The ingredients were mixed and filled into a gelatin capsule according to a conventional capsule preparation method to provide a capsule.

<1-4> Preparation of a Pill

| | |
|---|---|
| Arazyme | 1 g |
| Lactose | 1.5 g |
| Glycerin | 1 g |
| Xylitol | 0.5 g |

The ingredients were mixed and prepared into a pill according to a conventional method in such a manner that one pill has a weight of 4 g.

<1-5> Preparation of Granules

| | |
|---|---|
| Arazyme | 150 mg |
| Soybean extract | 50 mg |
| Glucose | 200 mg |
| Starch | 600 mg |

The ingredients were mixed and 100 mg of 30% ethanol was added thereto, followed by drying at 60° C. After formation of granules, the granules were filled into packaging.

<1-6> Preparation of Injectable Solutions

| | |
|---|---|
| Arazyme | 10 μg/ml |
| Diluted Hydrochloric Acid BP | until pH 7.6 |
| Injectable NaCl BP | up to 1 ml |

Arazyme was dissolved in a proper volume of injectable sodium chloride BP, pH of a solution produced was adjusted to pH 7.6 with diluted hydrochloric acid BP, and its volume was adjusted with injectable sodium chloride BP. After being sufficiently mixed, the solution was filled in a 5 ml and type I ampoule made from transparent glass, which was then molten such that the solution was packaged under the upper grid of air. An injectable solution was obtained by autoclaving the ampoule at 120° C. for 15 min or longer.

### Preparation Example 2

### Preparation of Food

The powders, tablets, capsules, pills and granules prepared in Preparation Example 1 may be applied to food. Foods containing Aranicola proteolyticus culture solution or arazyme isolated from the same were prepared as follows.

<2-1> Preparation of Flour Food

Aranicola proteolyticus culture solution or arazyme isolated from the same was added in an amount of 0.1 to 10 parts by weight to flour. Health enhancing foods such as bread, cake, cookies, crackers and noodles were prepared with the flour mixture according to a conventional method.

<2-2> Preparation of Soups and Gravies

Aranicola proteolyticus culture solution or arazyme isolated from the same was added in an amount of 0.1 to 10 parts by weight to soups and gravies. Health enhancing meat products, soups of noodles and gravies were prepared with the mixture by a conventional method.

<2-3> Preparation of Ground Beef

Health enhancing ground beef was prepared by adding Aranicola proteolyticus culture solution or arazyme isolated from the same in an amount of 10 parts by weight to ground beef according to a conventional method.

<2-4> Preparation of Dairy Products

Aranicola proteolyticus culture solution or arazyme isolated from the same was added in an amount of 0.1 to 10 parts by weight to milk. Various dairy products such as butter and ice cream were prepared with the milk mixture according to a conventional method.

<2-5> Preparation of Sun-Sik

Brown rice, barley, glutinous rice, adlay were pregelatinized, dried, and then roasted according to a conventionally known method. Then, the grains were prepared into powders with a grain size of 60 mesh by using a crusher.

Black soybean, black sesame, and perilla seeds were also steamed, dried and then roasted according to a conventionally known method. Then, the seeds were prepared into powders with a grain size of 60 mesh by using a crusher.

Aranicola proteolyticus culture solution or arazyme isolated from the same was vacuum-concentrated in a vacuum concentrator, and dried by spray and a hot wind dryer. Then the resulting dried product was prepared into dried powders with a grain size of 60 mesh by using a crusher.

The grains, the seeds and the dried powders of Aranicola proteolyticus culture solution or arazyme isolated from the same prepared above were blended with each other at the following ratios and prepared by a conventionally known method.

Grains (30 parts by weight of brown rice, 15 parts by weight of adlay, 20 parts by weight of barley),

Seeds (7 parts by weight of perilla seeds, 8 parts by weight of black soybean, 7 parts by weight of black sesame),

Dried powders of Aranicola proteolyticus culture solution or arazyme isolated from the same (1 part by weight),

Ganoderma lucidum (0.5 part by weight),

Rehmannia glutinosa (0.5 part by weight)

### Preparation Example 3

### Preparation of Beverages

Beverages containing Aranicola proteolyticus culture solution or arazyme isolated from the same were prepared as follows.

<3-1> Preparation of Health Beverages

Aranicola proteolyticus culture solution or arazyme isolated from the same (0.5 part by weight) was homogeneously mixed with liquid fructose (0.5 part by weight), oligosaccharide (2 parts by weight), sugar (2 parts by weight), table salt (0.5 part by weight), and water (75 parts by weight). Then, the mixture was sterilized instantly and filled into small containers such as glass bottles, pet bottles, etc. to prepare health beverages.

<3-2> Preparation of Vegetable Juice

Health enhancing vegetable juice was prepared by adding 0.5 g of Aranicola proteolyticus culture solution or arazyme isolated from the same to 1,000 ml and of vegetable juice such as tomato or carrot according to a conventional method.

<3-3> Preparation of Fruit Juice

Health enhancing fruit juice was prepared by adding 0.1 g of Aranicola proteolyticus culture solution or arazyme isolated from the same to 1,000 ml of fruit juice such as apple or grape according to a conventional method.

Arazyme produced from Aranicola proteolyticus of the present invention may be useful as a composition for prevention and treatment of arthritis by inhibiting the expression of TNF-α which is an inflammation-inducing factor and preventing the loss of proteoglycan and collagen from joint cartilage to prevent the progression of arthritis and protect joints.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A method for treating arthritis, comprising administering a pharmaceutically effective amount of arazyme to a subject in need thereof.

2. The method for treating arthritis as set forth in claim 1, wherein the arazyme is one of:
(a) a protein having the amino acid sequence represented by SEQ ID No: 1;
(b) a protein encoded by DNA comprising the coding region of the nucleotide sequence represented by SEQ ID No: 2;
(c) a protein having an amino acid sequence mutated from the amino acid sequence represented by SEQ ID No: 1 with substitution, deletion, insertion and/or addition of one or more amino acids therein and functionally identical to the protein having the amino acid sequence represented by SEQ ID No: 1; and
(d) a protein encoded by DNA hybridized with the DNA comprising the nucleotide sequence represented by SEQ ID No: 2 under the strict conditions and functionally equal to the protein having the amino acid sequence represented by SEQ. ID. NO: 1.

3. The method for treating arthritis as set forth in claim 1, wherein the arazyme is isolated from Aranicola proteolyticus culture solution.

4. The method for treating arthritis as set forth in claim 3, wherein the Aranicola proteolyticus is Aranicola proteolyticus HY-3 (Accession No: KCTC 0268BP).

5. The method for treating arthritis as set forth in claim 1, wherein the arthritis is degenerative arthritis (osteoarthritis), rheumatoid arthritis, ankylosing spondylitis, reactive arthritis, psoriatic arthritis, systemic lupus erythematosus, polymyositis, or polymyalgia rhematica.

6. The method for treating arthritis as set forth in claim 1, wherein the arazyme inhibits expression of TNF-α.

7. The method for treating arthritis as set forth in claim 1, wherein arazyme inhibits damage of joint cartilage.

8. The method for treating arthritis as set forth in claim 1, wherein the arazyme inhibits the loss of proteoglycan from joint cartilage.

9. The method for treating arthritis as set forth in claim 1, wherein the arazyme inhibits the loss of collagen from joint cartilage.

10. The method for treating arthritis as set forth in claim 1, wherein the arazyme is included in health food.

11. A method for preventing arthritis, comprising administering a pharmaceutically effective amount of arazyme to a subject in need thereof.

12.-18. (canceled)
